(19) United States
(12) Patent Application Publication
(10) Pub. No.: US 2024/0325688 A1
(43) Pub. Date: Oct. 3, 2024

(54) EXTENDABLE URINARY CATHETER PRODUCTS

(71) Applicant: Hollister Incorporated, Libertyville, IL (US)

(72) Inventors: Luis Martins, Ballina (IE); Vincent Naughton, Sligo (IE); William K. Arnold, Gurnee, IL (US); Damien Biggins, Ballina (IE); Jerome A. Henry, Castlebar (IE)

(21) Appl. No.: 18/575,146

(22) PCT Filed: Jun. 27, 2022
(86) PCT No.: PCT/US2022/035157

§ 371 Date: Dec. 28, 2023

Related U.S. Application Data
(60) Provisional application No. 63/218,148, filed on Jul. 2, 2021.

(51) Int. Cl. A61M 25/01; A61M 25/00

(52) U.S. Cl. CPC A61M 25/01; A61M 25/002; A61M 2025/0006; A61M 2025/0175; A61M 2210/1089

(57) ABSTRACT

A catheter product (200) comprising an extendable urinary catheter (20) including a proximal end section (22) and a distal end section (24) that are concentric and slide relative to one another, and a package (202) containing the extendable catheter (20) in a collapsed configuration. A retaining member (218) is associated with a proximal end portion (226) of an elongated body (204) of the package (202), the retaining member (218) holding the proximal end section (22) of the extendable urinary catheter (20) as the proximal and distal end sections (22.24) of the extendable urinary catheter (20) are pulled apart to slide relative to one another, thereby moving the extendable urinary catheter (20) into an extended configuration.

The present application claims the benefit of and priority to U.S. Provisional Patent Application No. 63/218,148, filed Jul. 2, 2021, which is hereby incorporated herein by reference.

### DESCRIPTION

### Technical Field

The present disclosure generally relates to extendable urinary catheter products that include catheters having a collapsed, storage configuration and an extended, use configuration, and more particularly, to products that include packaging that assists in moving the catheter from the collapsed configuration to the extended configuration.

### Background

Catheters are used to treat many different types of medical conditions and typically include an elongated catheter tube that is inserted into and through a passageway or lumen of the body. Urinary catheters and, in particular, intermittent urinary catheters are commonly used by individuals who suffer from certain abnormalities of the urinary system, such as urinary incontinence. With the advent of intermittent urinary catheters, individuals with problems associated with the urinary system can conveniently self-catheterize to drain the individual's bladder.

Individuals who suffer from urinary incontinence will self-catheterize several times a day. Thus, they are required to carry intermittent urinary catheters with them wherever they go. Accordingly, it is desirable for urinary catheters to have compact and portable configurations. It is also desirable for the catheters to be in configurations that allow the catheters to be discrete.

There remains a need for improved catheter assemblies that are compact, portable, and/or discreet.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a catheter product includes an extendable urinary catheter having a proximal end section and a distal end section that are concentric and slide relative to one another. In one embodiment, the urinary catheter may be telescopic. The extendable urinary catheter has a collapsed configuration and an extended configuration. The product also includes a package containing the extendable catheter in the collapsed configuration. The package includes a sidewall defining an elongated body having a cavity containing the catheter, wherein the sidewall extends over a majority of the proximal end section of the catheter and a majority of the distal end section of the catheter. The product further includes a retaining member associated with a proximal end portion of the body of the package. The retaining member holding the proximal end section of the extendable urinary catheter as the proximal and distal end sections of the extendable urinary catheter are pulled apart to slide the proximal and distal end sections relative to one another, thereby moving the extendable urinary catheter into the extended configuration.

In another aspect, a catheter product includes a telescope urinary catheter having a proximal end section and a distal end section that are concentric and slide relative to one another. The extendable urinary catheter has a collapsed configuration and an extended configuration. The product also includes a package defining a body that contains the extendable catheter in the collapsed configuration. The product also includes retaining member releasably engaged to the proximal end section of the catheter. The retaining member is movable axially within the body. The product further includes a stop located in the body of the package. The retaining member engaging the stop as the catheter is pulled from the body of the package to move the catheter into the extended configuration.

In yet another embodiment, a catheter product includes an extendable urinary catheter including a proximal end section and a distal end section that are concentric and slide relative to one another. The extendable urinary catheter having a collapsed configuration and an extended configuration. The product further including a package having a body defining a cavity that contains the extendable catheter in the collapsed configuration. The product also includes one or more flexible retaining members within the body. The flexible retaining members including one or more flexible projections having a free end engaging at least one eyelet in the proximal end section of the extendable urinary catheter and holding the proximal end section of the extendable urinary catheter as the proximal and distal end sections of the extendable urinary catheter are slid relative to one another to move the extendable urinary catheter into the extended configuration.

### BRIEF DESCRIPTION OF FIGURES

FIG. 1 is a perspective view of an exemplary urinary catheter that may be used in a catheter product in accordance with the present disclosure, the catheter being shown in the collapsed, storage configuration;

FIG. 2 is a perspective view of the urinary catheter of FIG. 1, shown in the extended, use configuration;

FIG. 3 is a perspective view of a urinary catheter product;

FIG. 4 is a perspective view of the urinary catheter product of FIG. 3, shown with the catheter being partially removed from the package;

FIG. 5 is a perspective view of the urinary catheter product of FIG. 3, showing the catheter being moved into the extended configuration as the catheter is removed from the package;

FIG. 6 is a partial cross-sectional view of the extendable catheter of FIG. 1, showing one alternative of interlocking mechanism in the unlocked state;

FIG. 7 is a side perspective view of the interlocking member shown FIG. 6;

FIG. 8 is a rear perspective view of the interlocking member of FIG. 6;

FIG. 9 is a front perspective view of the interlocking member of FIG. 6;

FIGS. 10-14 are cross-sectional views illustrating the locking mechanism moving from the initial position to the locked position;

FIG. 15 is a partial cross-sectional view of one alternative of a catheter product in accordance with the present disclosure, shown with the catheter in a collapsed configuration within the package;

FIG. 16 is a cross-sectional view of the shuttle of the catheter product shown in FIG. 15;

FIG. 17 is a cross-sectional view of the urinary catheter product of FIG. 15, shown with the catheter being removed from the package and being moved into the extended configuration;

FIG. 18 is a cross-sectional view of the urinary catheter product of FIG. 15, shown after the catheter has been moved into the extended configuration and removed from the package;

FIG. 19 is a side view of another alternative of a catheter product in accordance with the present disclosure;

FIG. 20 is a side view of the catheter product of FIG. 19;

FIG. 21 is a side view of the catheter product of FIG. 19;

FIG. 22 is a side view of the catheter product of FIG. 19;

FIG. 23 is a side view of another alternative of a catheter product in accordance with the present disclosure;

FIG. 24 is a side view of the catheter product of FIG. 23;

FIG. 25 is a perspective view an alternative embodiment of a collar in accordance with the present disclosure;

FIG. 26 is a side view showing the collar in the package and engaged with the eyelets of the catheter;

FIG. 27 is a perspective exploded view of another alternative of a catheter product in accordance with the present disclosure, shown with the catheter in a collapsed configuration prior to being assembled with the package;

FIG. 28 is a cross-sectional view of the urinary catheter product of FIG. 27;

FIG. 29 is a perspective the catheter product of FIG. 27, illustrating assembly of the catheter product;

FIG. 30 is a cross-sectional exploded view of another alternative of a catheter product in accordance with the present disclosure, shown with the catheter in a collapsed configuration prior to being inserted into the body of the package;

FIG. 31 is a perspective view of a collar of the product shown in FIG. 30;

FIG. 32 is a cross-sectional view of the of the product of FIG. 30, shown with the catheter and retaining member being positioned within the package;

FIG. 33 is a partial cross-sectional view of one alternative of a catheter product in accordance with the present disclosure;

FIG. 34 is a partial cross-sectional view the catheter product of FIG. 33;

FIG. 35 is a partial cross-sectional view the catheter product of FIG. 33, shown with the catheter moved into the extended configuration;

FIG. 36 is a side view of another alternative of a catheter product in accordance with the present disclosure;

FIG. 37 is a cross-sectional view of the catheter product of FIG. 36;

FIG. 38 is a front perspective view of the collar shown in FIG. 36;

FIG. 39 is a top perspective view of the collar shown in FIG. 36;

FIG. 40 is a partial cross-sectional view of the catheter product of FIG. 36;

FIG. 41 is a partial cross-sectional view of the package of the catheter product of FIG. 36 shown without the catheter and collar, taken along line 41-41 of FIG. 37;

FIG. 42 is a cross-sectional view of the package of the catheter product of FIG. 36 shown without the catheter and collar, taken along line 42-42 of FIG. 41;

FIG. 43 is a cross-sectional view of the package of the catheter product of FIG. 36 shown without the catheter and collar, taken along line 43-43 of FIG. 41;

FIG. 44 is a cross-sectional view of the package of the catheter product of FIG. 36 with the catheter and collar within the package, taken along line 44-44 of FIG. 37;

FIG. 45 is a cross-sectional view of the package of the catheter product of FIG. 36 with collar shown in a first position, taken along line 45-45 of FIG. 37; and

FIG. 46 is a cross-sectional view of the package of the catheter product of FIG. 36 with collar shown in a second position, taken along line 46-46 of FIG. 37.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

The present disclosure is directed to urinary catheter products wherein the urinary catheter has a collapsed, storage configuration and an extended, use configuration. The catheter product includes a package having a retaining member that assists with moving the urinary catheter from the collapsed configuration to the extended configuration, as the catheter is removed from the package.

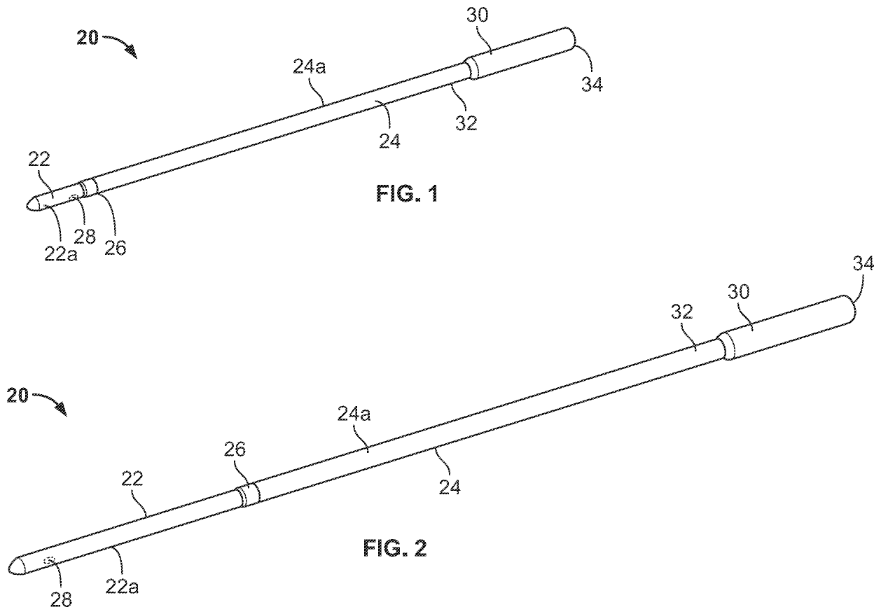

FIGS. 1 and 2 illustrate one exemplary embodiment of a urinary catheter 20 for use in the catheter products of the present disclosure. The catheter 20 may be a telescoping catheter that includes a proximal end section 22 and distal end section 24. The proximal and distal end sections 22 and 24 may be in telescopic relation to one another and/or connected to each other by a telescopic joint. In the illustrated embodiment, the proximal end section 22 includes a proximal tube 22a and the distal end section includes a distal tube 24a. The proximal end section 22 and the distal end section 24 are concentric and slide relative to one another. In the illustrated embodiment, the proximal section 22 is located and slidable within a lumen of the distal section 24. In other alternatives, the distal section 24 could be located and slidable within a lumen of the proximal section 22.

FIG. 1 shows the catheter 20 in a collapsed, storage configuration. In the collapsed configuration, a majority of the proximal end section 22 and a majority of the distal end section 24 are overlapping one another. In the illustrated embodiment, the majority of proximal tube 22a is located within a lumen of the distal tube 24a. The catheter 20 can be moved from the collapsed configuration shown in FIG. 1 to the expanded configuration shown in FIG. 2. To move the catheter 20 into the expanded configuration, the proximal end section 22 and distal end section 24 are slid relative to one another in an extending telescoping fashion. As shown in FIG. 2, the axial length of the catheter 20 in the extended configuration is larger than the axial length of the catheter 20 in the collapsed configuration. When in the extended configuration, the catheter 20 is ready for use. In use, the telescopic joint 26 joining the proximal and distal end sections 22 and 24 may or may not be configured to be insertable into the urethra.

The proximal end section 22 of the catheter 20 includes one or more eyelets 28 for draining urine from the bladder. The eyelets 28 are in communication with a drainage lumen (not shown) of the proximal end section 22. The drainage lumen of the proximal end section 22 is in communication with the lumen (not shown) of the distal end section 24. A drainage member 30 may be located at the distal end 32 of the distal end section 24. The drainage member 30 may be a funnel or connector that has a drainage outlet 34 for directing urine drainage out of the catheter 20. The drainage member 30 may also serve as a handle or gripping member for the user. The gripping member may be gripped by the user to apply a pulling force to move the catheter 20 from the collapsed configuration to the extended configuration.

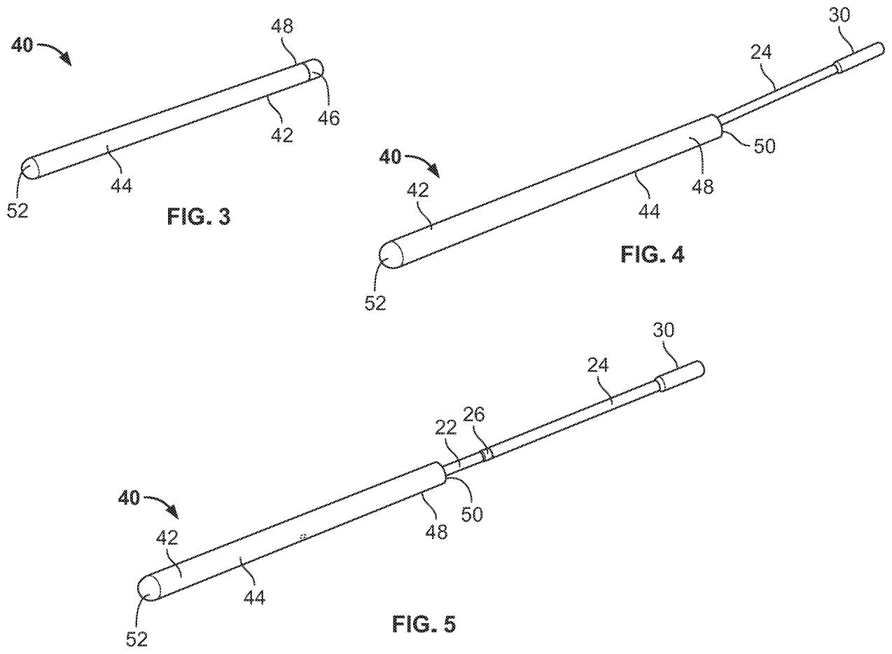

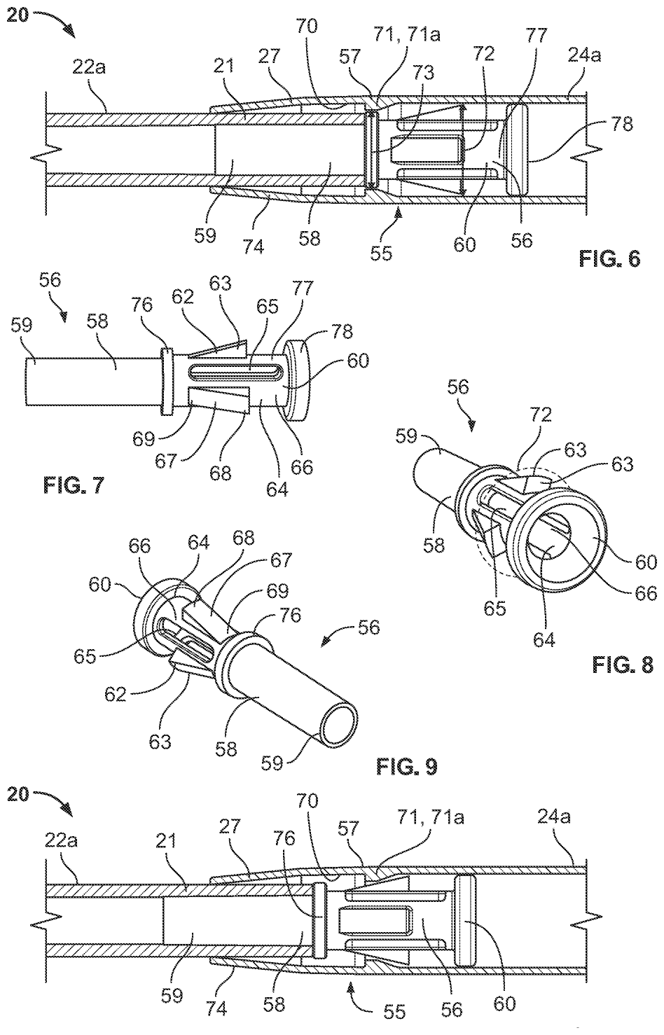

FIGS. 3-5 illustrate one embodiment of a catheter product 40 that includes a package 42 and catheter 20, or any other suitable extendable catheter. The package 42 includes an elongated body 44 having a sidewall that defines a cavity (shown in FIGS. 6, 11, 15 and 17) for containing the catheter 20 when the catheter is in the collapsed configuration. The cavity body 40/package 42 may include other elements as well, such as a hydration fluid (liquid and/or vapor) when the urinary catheter is a hydrophilic a catheter, or a gel when the urinary catheter is a gel lubricated catheter. In one alternative, the proximal and distal end sections 22 and 24 of the catheter 20, in the collapsed configuration, are located within the cavity wherein the sidewall of the body 44 extends over the entire lengths of both proximal and distal end distal sections 22 and 24. In another alternative, the sidewall of the body 44 extends partially or at least partially over the proximal and distal end sections 22 and 24 of the catheter 20. Optionally, the package 42 includes a distal removable cap or lid 46 (FIG. 3) removably attached to the distal end 48 of the body 44 to cover the distal end opening 50 of the body 44. The body 44 has a closed proximal end 52. The package 42 may provide a sterile package that is gas and/or liquid impermeable. In one alterative, the cap 46 at least partially covers and/or has a cavity (not shown) in which at least a portion of the drainage member 30 is located. In another alternative, the drainage member 30 may serve as the cap closing the package 42. For example, the drainage member 30 may mate with the distal end 48 of the body to close opening 50, wherein the drainage member 30 includes a removable seal over the drainage opening 34.

In use, the cap 46 of the package 42 is removed from the body 44 to expose the drainage member 30 extending out from an opening 50 in the distal end 48 of the body 44. The user grasps the drainage member 30 with one hand or part of the user's body (e.g., between arm and side or between knees) and grasps the body 44 of the package 42 with the other hand or part of the user's body. The user then pulls the catheter 20 and body 44 of package 42 apart. As will be discussed in more detail below, the package includes a retaining member (e.g., shown in FIGS. 6, 11, 15 and 17) that holds or retains at least a portion of the proximal end section 22 of the catheter 20 within the body 44 of the package 42. Because the retaining member retains at least a portion of the proximal section 22 of the catheter 20 with in the body 44, the force applied from pulling the catheter 20 and body 44 apart moves the proximal end section 22 and the distal end section of the catheter 20 relative to each other to place the catheter 20 into the extended configuration. Once the catheter 20 is in the extended configuration, continued pulling of the catheter 20 away from the body 44 of the package 42 releases the catheter 20 from the retaining member. Thereby, fully removing the catheter 20 from the body. The catheter 20 is now removed from the package 42, in the extended configuration and ready for use.

FIGS. 6 and 10-14 illustrate an alternative interlocking mechanism 55 that may be used in extendable catheter 20 to lock the proximal tube 22a and the distal tube 24a in the extended configuration. Alternatively, the interlocking mechanism may be any other interlocking mechanism suitable for locking the catheter 20 into the extended configuration. FIG. 6 shows the catheter 20 in the collapsed configuration and the interlocking mechanism 55 in an initial or unlocked state. FIG. 14 shows the catheter 20 in the extended configuration and the interlocking mechanism 55 in the locked state. In the locked state, the interlocking mechanism 55 has sufficient strength to prevent the catheter 20 from returning to the collapsed configuration during use. The interlocking mechanism 55 also prevents the proximal tube 22a and distal tube 24a from separating.

The interlocking mechanism 55 includes a first interlocking member 56 and a second interlocking member 57, which engage each other to lock the catheter 20 in an extended configuration or position. The first interlocking member 56 is associated with the distal interlocking end portion 21 of the proximal tube 22a. In the illustrated embodiment, the first interlocking member 56 has a body 58 that includes a proximal end portion 59 and a distal end portion 60. The body 58 may be generally tubular with a longitudinal axis “X” (FIG. 14) and a lumen 61 (FIGS. 12 and 14) therethrough. The proximal end portion 59 of the first interlocking member 56 is connected to the distal end portion 21 of proximal tube 22a. In one alternative, the distal end portion 21 of the proximal tube 22a is over-molded over the proximal end portion 59 of the first interlocking member 56. In another option, the proximal end portion 59 of the first interlocking member 56 is embedded in the distal end portion 21 of the proximal tube 22a. In yet another alternative, the distal end portion 21 of the proximal tube 22a is friction or heat fit over the proximal end portion 59 of the first interlocking member 56.

Referring to FIGS. 7-9, the first interlocking member 56 includes one or more latches 62 extending radially outward from the wall 64 of the body 58. In the illustrated embodiment, the latches comprise fins 63 which may have a generally triangular cross-section and/or have the shape of a triangular prism. The wall 64 at the distal end portion 60 of the first interlocking member 56 is flexible and elastic. In the illustrated embodiment, the wall 64 of the distal end portion 60 includes a plurality of longitudinal openings 65, such as cavities, slots, or cut-outs, which aid in allowing the wall 64 to flex. The flexibility and elasticity of the wall may be varied by varying the material of the wall, the thickness of the wall and the size and shape of the opening. The structures of the wall 64 between the openings 65 define columns 66 which are flexible and elastic. The columns 66 may be compressible inward and then return to their initial state. For example, when force is applied to the columns, they may be compressed inward toward the longitudinal axis.

The fins 63 extend longitudinally along the columns 66 and radially outwardly from the columns 66. In the illustrated embodiment, the fins 63 have an outer surface 67 that is ramped or angled with respect the surface of the wall 64, the columns 66 and/or the longitudinal axis “X” of the body 59. The outer surface 67 angles radially inwardly toward the surface of the wall 64 from distal end 68 of the fin to the proximal end 69 of the fin (FIGS. 7 and 9).

Referring back to FIGS. 6 and 10-14, the second interlocking member 57 may be defined by or at the proximal end portion 27 of the distal tube 24a. The inner surface 70 of the proximal end portion 27 may include a keeper 71. The keeper 71 comprises one or more projections 71a extending into the lumen of the distal tube 24a. The projection(s) 71a may be integral with the wall of the distal tube 24a. In one alternative, the projection 71a may be a continuous, circumferential projection. In another alternative, the projection(s) 71a may be a plurality of projections. The projection(s) 71a may be integral with the distal tube 24a. Alternatively, the projection(s) 71a may an insert or a component of an insert that is located within the proximal end of the distal tube 24a.

The maximum combined outer radial boundary 72 defined by fins 63 is larger than the maximum combined inner radial boundary 73 defined by the projection(s) 71a of the keeper 71 (FIGS. 6 and 8). Furthermore, the inner surface 70 and outer surface 74 of the proximal end portion 27 of the distal tube 24a may be tapered radially inwardly in the proximal direction. For example, the outer surface 74 of the proximal end portion 27 of the distal tube 24a may be tapered to form a smooth transition with the outer surface 75 of the proximal tube 22a. Furthermore, the inner surface 70 of the proximal end portion 27 of the distal tube 24a may be tapered so as to have a diameter that is smaller than an outer diameter of a projection 76 of the body 58 of the first interlocking member 56. In one alternatively, the body includes a circumferential projection 76 extending radially from the body 58. The circumferential projection 76 has an outer diameter larger than at least a portion of the tapered proximal end 27 of the distal tube 24a. Contact between the tapered distal end 27 and the circumferential projection 76 prevent the tubes 22a, 24a from separating. In one alternative, distal tube 24a may be made from component parts wherein the tapered proximal end 27 and the projection(s) 71a may be components of an insert that is connected to the remaining section of the distal end tube 24a. Furthermore, the terminal distal end 78 of the body 58 may have a larger cross-section than an adjacent section 77 of the distal end portion 60 (FIGS. 6 and 7). For example, the distal end portion 60 may include a large flange that is has a larger outer boundary than the adjacent section 77. The terminal distal end 78 contacts the inner surface of the distal tube 24a. This may assist in stabilizing the body 58 within the distal tube 24a and serve as an additional safety stop that prevents the tubes 22a, 24a from separating.

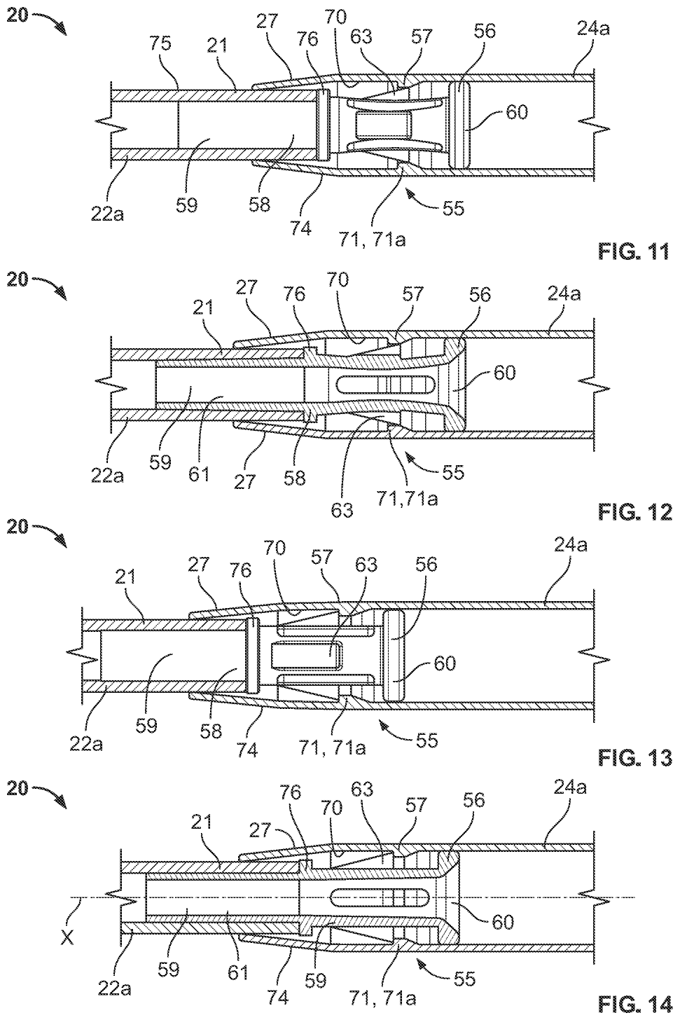

The extendable catheter 20 is stored and packaged (not shown) in the collapsed configuration shown in FIG. 6. After the user opens the package, the user moves the proximal and distal tubes 22a, 24a away from each other, moving the proximal tube 22a out of the lumen of the distal tube 24a. The maximin combined diameter of the fins 63 of the latch is larger than the combined inner diameter of the keeper's projection(s) 71a. As the user continues to move the proximal and distal tubes 22a, 24a apart, the first interlocking member 56 contacts the projection(s) 71a of the keeper 71 of the second interlocking member 57. For example, the fins 63 of the first interlocking member 56 contact the projection(s) 71a of the second interlocking member 57. The ramped or angled outer surfaces 67 of the fins 63 engages the projection(s) 71a (FIG. 10). As the tubes 22a, 24a continue to be moved apart and the ramped outer surfaces 67 of the fins 63 move along the projection(s) 71a, force is placed on the columns 66. The columns 66 flex or collapse inward allowing the fins 63 to pass by the projection(s) 71a (FIGS. 11 and 12). After the distal ends 68 of the fins 63 pass the projection(s) 71a, the columns 66 move back toward their initial state and the distal ends 68 of the fins 63 engage the projection(s) 71a to lock the catheter 20 in the extended configuration (FIGS. 13 and 14). Furthermore, the circumferential projection 76 of the body 58 of the first interlocking member 56 contacts the tapered inner surface 70 of the proximal end 27 of the distal tube 24a, which acts as a stop or stop mechanism to prevent the tubes 22a, 24a from separating. The distance between the latch and the stop is such that there is minimal relative movement between the two tubes. The user then preforms catheterization with the catheter by inserting the catheter into the urethra. During catheterization, the proximal end 27 of the distal tube 24a may have an insertable portion that may be inserted into the urethra.

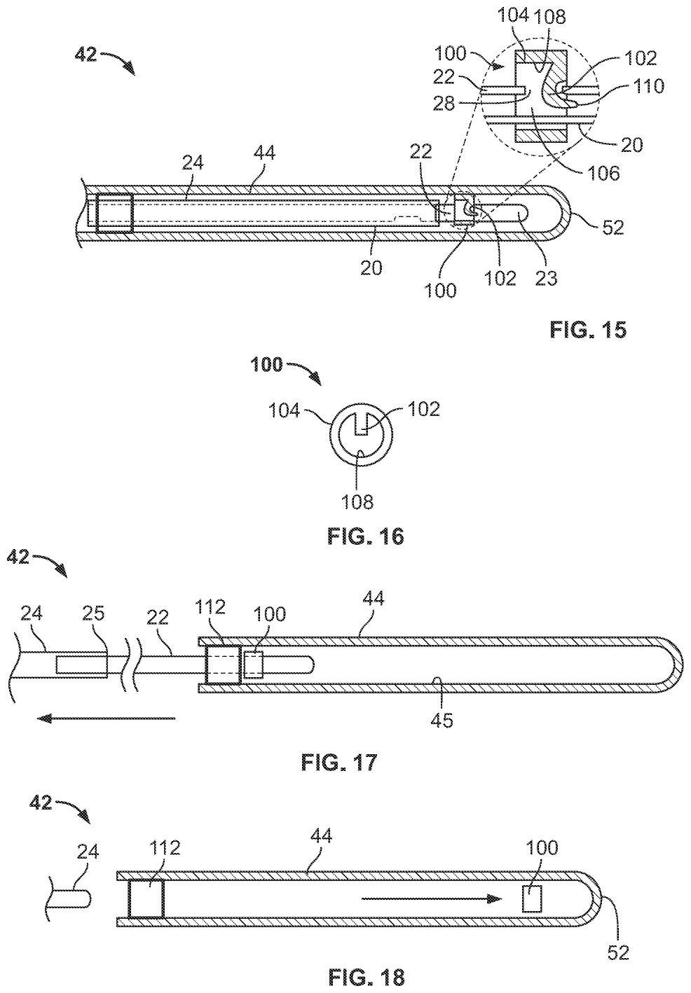

FIGS. 15-18 illustrate one alternative retaining member 100 that may by associated with the package 42. The retaining member 100 includes one or more flexible projections 102 that engages at least one eyelet 28 of the catheter 20. In the illustrate embodiment, the retaining member 100 includes a sidewall 104 that defines a collar or shuttle having a lumen 106 that receives the proximal end section 22 of the catheter 20. The illustrated retaining member 100 is shown as a ring shaped collar. In another alternative, the sidewall 104 defining the collar may not have a full ring-shape. For example, the sidewall 104 may define a C-shaped collar.

The projection 102 extends from the inner surface 108 of the collar and includes a free-end 110 that is inserted into and engages the eyelet 28 of the catheter. As shown in FIG. 15, the proximal end section 22 of the catheter 20 is coaxial and positioned within the distal end section 24 of the catheter 20, wherein a proximal end 23 of the proximal end section extends out of a proximal opening 25 of the distal end section 24. The retaining member 100 is positioned with the collar about the proximal end 23 of the proximal section 22. The projection 102 extends into the eyelet 28 of the catheter 20 to releasably attach the retaining member 100 to the proximal end section 22 of the catheter 20. Movement of the retaining member 100 is not fixed axially relative to the package 42, such that the retaining member 100 may move axially within the package 42. For example, the retaining member 100 may not be attached to the package 42 or it may be attached but axially moveable relative to the package 42. For instance, the retaining 100 may be mated to a track on the inner surface of the package 42 that allows movement of the retaining member 100 axially relative to the package 42.

The projection 102 of the retaining member 100, as well as the other projections disclosed herein, may be any suitable projection that has sufficient rigidity to maintain engagement with the eyelet 28 and withstand the deflection force that arises from the catheter 20 is being moved from the collapsed configuration to the extended configuration and sufficiently flexibility to flex or bend so that it disengages from the eyelet 28 after the catheter 20 has been extended and is being pulled from the package 42 for use. Alternatively, or in addition to, the material of the catheter 20 defining the eyelet 28 may have sufficient rigidity to maintain engagement with the projection 102 while the catheter 20 is being moved from the collapsed configuration to the extended configuration and sufficiently flexibility to flex or bend to disengage from the projection 102 after the catheter 20 has been extended and is being pulled from the package 42. Furthermore, the flexibility/rigidity of the projection and the material of the catheter could be tuned relative to one another for extension and removal from the package.

In the illustrated alternative of FIGS. 15 and 16, the projection 102 of the retaining member 100 has a generally hooked-shape. The projection 102, and the other projections described herein, could have any suitable shape that engages the eyelet 28 of the catheter 20. For example, the projection 102 could be post. Furthermore, the projection 102 could vary in cross-sectional thickness or diameter. For example, the projection 102 could have a large cross-sectional width at one end and a smaller cross-sectional width along the projection or at the other end of the projection. For example, the projection 102 could be cone or needle-shaped.

As shown in FIG. 17, the catheter 20 is moved distally out of the body 44 of the package 42. For example, the user may open the package 42 and then grasp the body 44 of the package 42 and the distal end section 24 of the catheter 20. The user then pulls the distal end section 24 of the catheter 20 away from the body 44 of the package 42. As the user pulls, the proximal end section 22 of the catheter 20 and the retaining member 100 move distally within the body 44 until the retaining member 100 contacts a stop 112 associated with the inner surface 45 of the body 44. In the illustrated embodiment, the stop is associated with the distal end 48 of the body 44 of the package 42, but the stop could also be associated with the inner surface 45 of any other portion of the body. In one alternative the stop 112 could be integral with the body 44 or it may be inserted into and engaged with the inner surface 45 of the body. For example, the stop 112 could be a ridge or lip associated with the inner surface 45 of the body 44. Such a stop 112 may be formed in an injection molding process that forms the body 44. Alternatively, the stop 112 may be a gromet positioned within the cavity of the body 44. When the retaining member 100 contacts the stop 112, it retains the proximal 22 of the catheter within or attached to the body 44 of the package 42. Continued pulling of the distal end section 24 of the catheter 20 results in the distal and proximal end sections 22 and 24 of the catheter 20 sliding relative to one another, thereby telescoping the catheter 20 and moving it in the extended configuration shown in FIG. 2. After the catheter 20 has been moved and locked into the extended configuration, continued pulling of the catheter 20 releases the catheter 20 from the retaining member 100. As mentioned above, the projection 102 of the retaining member 100 may be a flexible projection that has sufficient rigidity so as to maintain engage with the eyelet 28 while the catheter 20 is being pulled to place the catheter 20 in the extended and locked configuration. The flexible projection 102 also has sufficient flexibility so that the projection 102 bends and disengages from the eyelet 28 of the catheter 20 under continued pulling force after the catheter 20 has been moved into the extended configuration. Referring to FIG. 18, after the retaining member 100 disengages from the catheter 20, the retaining member 100 remains in the body 44 of the package 42 between the stop 112 and the distal end 52 of the body 44.

The catheter product includes an extendable catheter in a collapsed configuration. The extendable catheter is moved into an expanded configuration when subjected to a first pulling force to slide the distal end section of the catheter relative to the proximal end section of the catheter. The first pulling force also moves the locking mechanism in the extended configuration, thereby locking the catheter in the extended configuration. The flexible projection is configured to retain engagement with the eyelet of the catheter when the catheter is subjected to this first catheter expanding pulling force. Once the catheter has been moved into and locked in the expanded configuration, the catheter is subjected to a second pulling force, which is greater than the first pulling force, to disengage the catheter from the retaining member and therefore remove the catheter from the body of the package. The flexible projection is configured to bend and disengage the eyelet when the catheter is subjected to this second greater removal pulling force.

The holding force of the engagement between the retaining member and the distal end of the catheter and/or the eyelet is larger than the force needed to move the catheter into the expanded configuration and/or lock the catheter in the expanded configuration. Additionally, the holding force is smaller than the removal force to remove the catheter from the package for use. The holding force is the force needed to overcome to disengage the retaining member from the eyelet or distal end of the catheter.

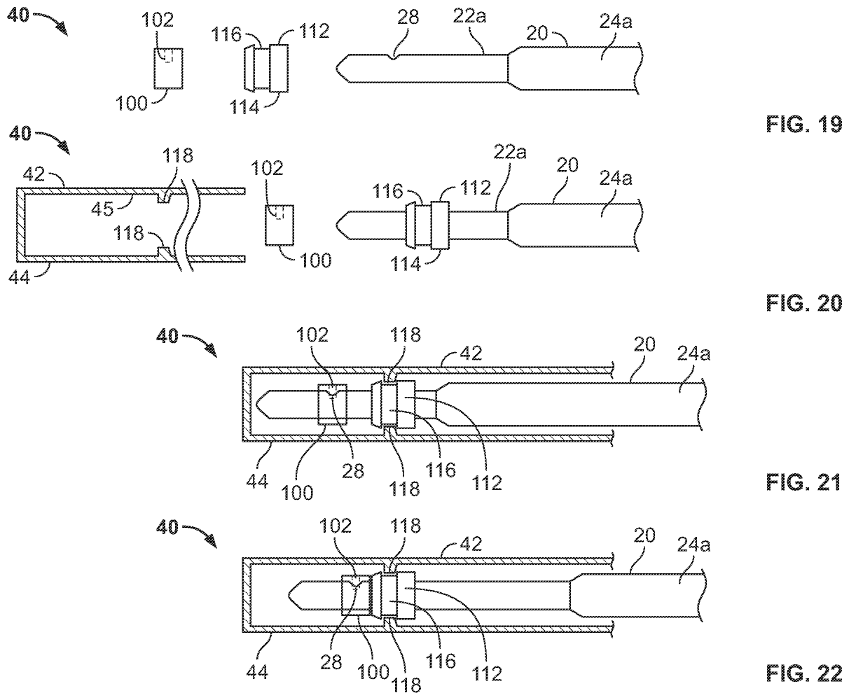

FIGS. 19-22 illustrate one alternative for catheter product 40 and a method of assembling the catheter product 40. Referring to FIG. 19, the retaining member 100 and the stop 112 are separate from the catheter 20. The stop 112 may be a gromet. For example, the stop 112 has a generally tubular shape with a lumen therethrough for receiving the proximal tube 22a of the catheter 20. The outer surface 114 of the stop 112 includes one or more indents 116. The one or more indents 116 may be for example one or move groves. The inner diameter of the lumen of the stop 112 is larger than the outer diameter of the proximal tube 22a and smaller than the outer diameter of the distal tube 24a. Accordingly, the stop 112 is movable over the proximal tube 22a but does not fit over the distal tube 24a.

Referring to FIG. 20, to assembly the catheter product 40, the proximal tube 22a is inserted into the lumen of the stop 112 and the stop is moved distally along proximal tube 22a. Referring to FIG. 21, the proximal tube 22a is inserted into the lumen of the retaining member 100 and the projection 102 of the retaining member 100 engages the eyelet 28 of the catheter 20. The proximal tube 24a of the catheter 20 is then inserted into the package 42/body 44 of the package. The catheter 20 is inserted until the indent(s) 116 engage a portion of the inner surface 45 of the body 44 to hold the stop 112 in place. The inner surface 45 of the body 44 includes one or more projections 118 that engage the indent(s) 116 to hold the stop 112 in place. In other alternatives, the stop 112 may be held in place in any suitable manner, such as a snap fit, friction fit, welding, solvent bonding or adhesive. Referring to FIG. 22, as the distal tube 24a is pulled from the package, the retaining member 100 abuts the stop 112, which holds the retaining member in place while the catheter is moved into the extended configuration similar to above.

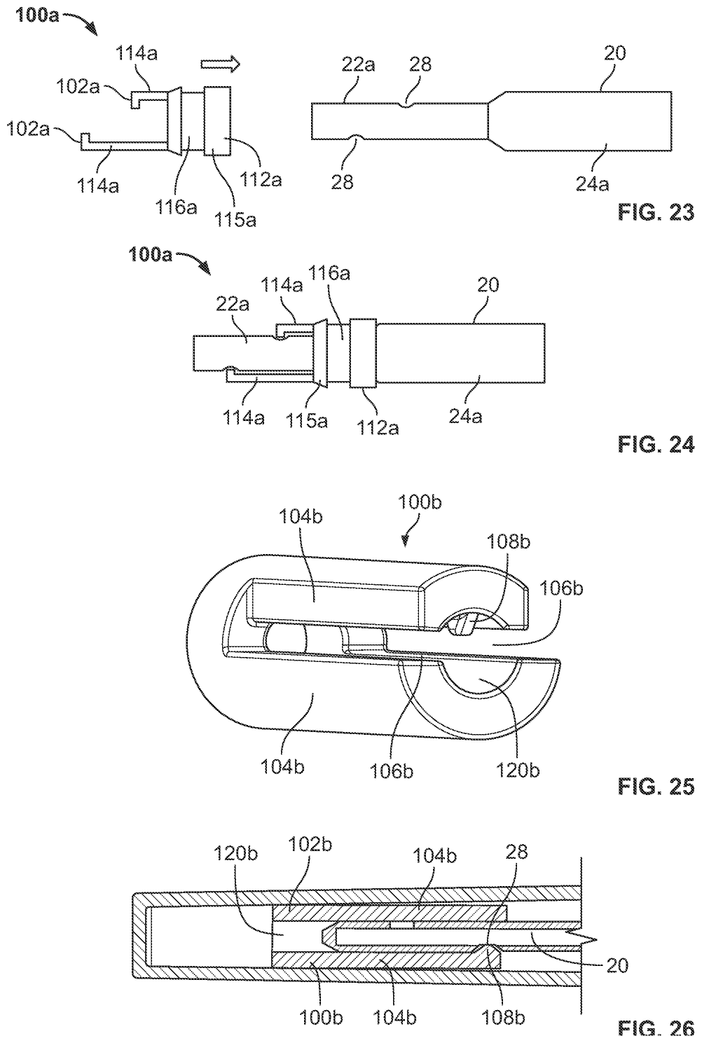

Turing to FIG. 23, there is shown an alternative retaining member 100a, which is configured to engage the inner surface 45 of the package body 44 (FIG. 20) to hold the retaining member in place. The retaining member 110a has one or more retaining arms 114a extending from a gromet portion 112a. The gromet portion 112a has a generally tubular shape that includes a lumen therethrough for receiving the proximal tube 22a of the catheter 20. The outer surface 115a of the gromet portion 112a includes one or more indents 116a. The one or more indents 116a may be for example one or move groves. The inner diameter of the lumen of the gromet portion 112a is larger than the outer diameter of the proximal tube 22a and smaller than the outer diameter of the distal tube 24a. Accordingly, the gromet portion 112a is movable over the proximal tube 22a but does not fit over the distal tube 24a.

The one or more retaining arms 114a include a projection 102a for engaging the eyelet 28 of the catheter 20. The proximal tube 22a of the catheter is inserted through the lumen of the gromet portion 112a. The projections 102a engage the eyelets to hold the proximal tube 22a of the catheter. The retaining member 100a may be inserted into the package body 44 so that the indents 116a engage one or more projections 118 of the inner surface 45 of the package body 44. The retaining member 100a may be inserted first into the package body 44 and then the catheter 20 may be inserted into the package body 44 and through the lumen of the retaining members gromet portion 112a. The eyelets may then engage the projections 102a of the arms 114a. Alternatively, the retaining member 100a can be placed on the catheter's proximal tube 22a and the catheter 20 having the retaining member 100a thereon can be inserted into the package body 44 until the gromet portion 112a engages the projections 118.

FIGS. 25 and 26 illustrate an alternative retaining member 100b, which can be one that is held in place by a stop or can itself engage the inner surface 45 of the package body 44. The retaining member 100b has a generally tubular shape including a lumen 120b therethrough. The retaining member 100b includes a base 102b with one or more arms 104b extending from the base 102b. The illustrated embodiment includes the base 102b at the proximal end of the retaining member 100b and two arms 104b extending in a distal direction from the base. Slots 106b separate the arms 104b. In the illustrated embodiment, the arms 104b having an arcuate cross-section. The radius of the arcuate may the same of different. One or more of the arms 104b may include a projection 108b that engages an eyelet 28 of the catheter 20, as shown in FIG. 26.

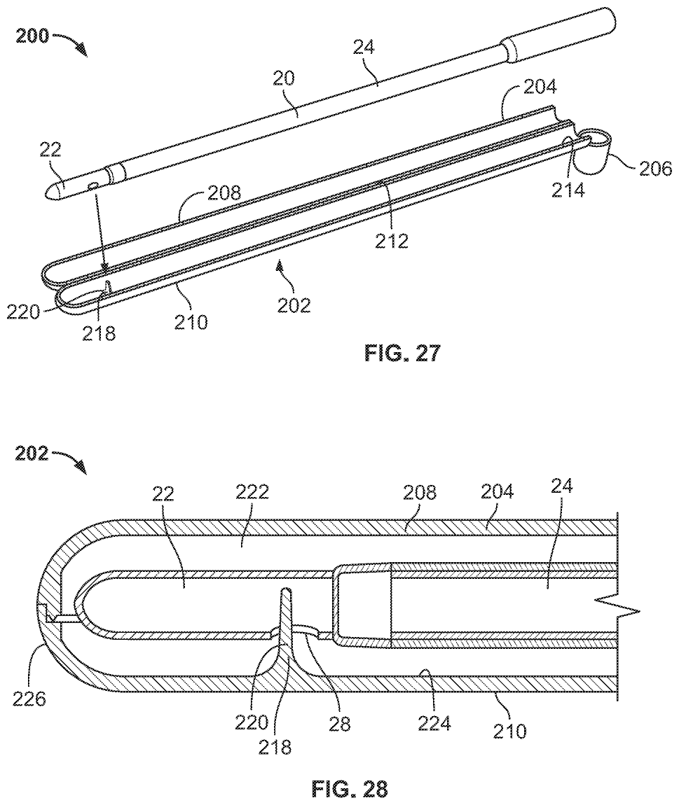

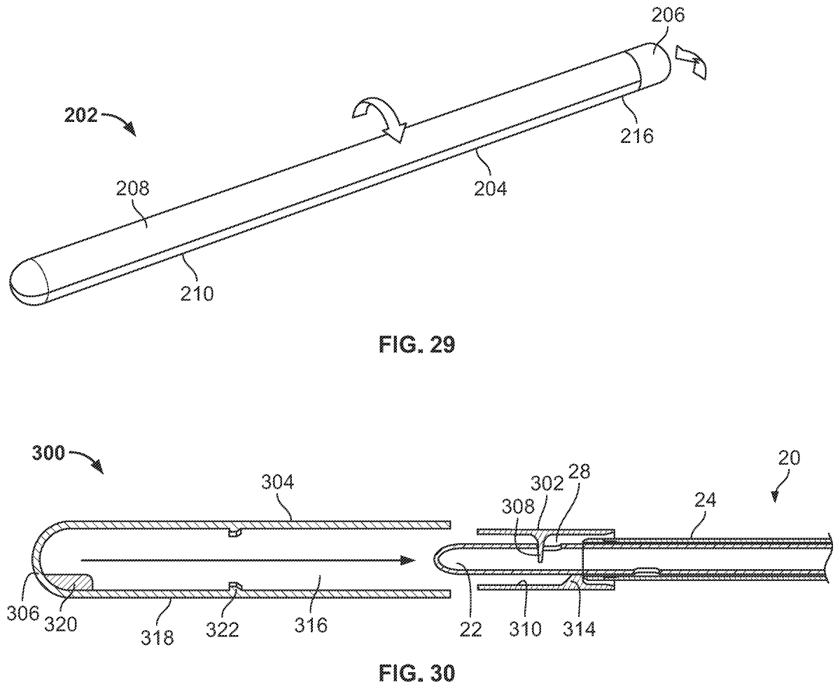

FIGS. 27-29 illustrate another alternative catheter product 200. The product 200 includes an expandable catheter 20 and a package 202. The package 202 includes a body 204 and a cap 206. The body 204 has a generally clamshell configuration that includes a top portion 208 and a bottom portion 210. In the illustrated embodiment, the top and bottom portions 208 and 210 are connected by a hinge 212. In an alternative, the top and bottom portions 208 and 210 may be unconnected discrete components that are not hingedly connected. Optionally, the cap 206 may be connected to one of the top and bottom portions 208 and 210 by a hinge 214. Alternatively, the cap 206 may be a separate and discrete component that is releasably attached to the distal end 216 of the body 204 to close the package 202 as shown in FIG. 29.

Referring to FIGS. 27 and 28, the package 202 may also include a retaining member 218 that engages the eyelet 28 of the catheter 20. The retaining member 218 includes one or more projections 220 that project into the cavity 222 defined by the package body 204. In the illustrated embodiment, the projection 220 projects in a direction from an inner surface 224 of one of the first and second portions 208 and 210. The projection 220 is located at or near the proximal end 226 of the package 202 and may have any suitable shape or flexibility as described above. In the illustrated embodiment the projection 220 is a post having a generally elongated cone or needle shape.

Referring to FIGS. 27-29, to package the catheter product, the top and bottom portions 208 and 210 are open/separated, as shown in FIG. 27. Referring to FIG. 28, the catheter 20 is placed in the body 204 of the package 202 so that the one or more projections 220 engages at least one eyelet 28 of the catheter 20. Referring to FIG. 29, the clam shell body 204 is closed so that the top and bottom portions 208 and 210 are attached to each other to define the cavity 222 (FIG. 28) that contains the catheter 20. The cap 206 is placed on the distal end 216 of the body 204 to close the package 202. To use the catheter product, the cap 206 is removed, and the distal end section 24 of the catheter 20 is pulled in a manner similar to that shown in FIGS. 3-5, to extend and remove the catheter 20 from the package 202.

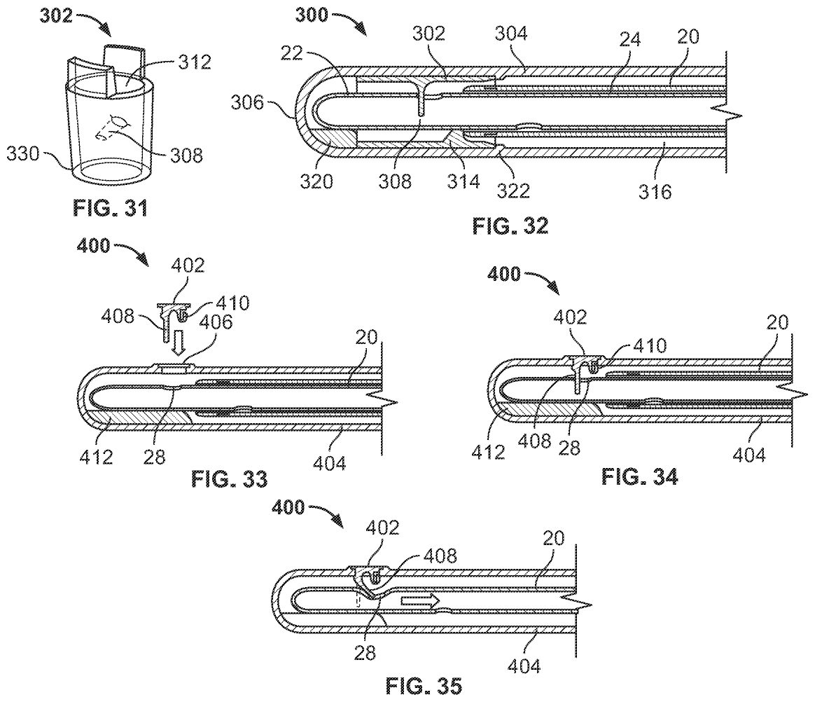

FIGS. 30-32 illustrate another alternative package 300 having a retaining member 302. In this embodiment, the retraining member 302 may be a collar that is inserted into the body 304 of the package 300 and attached to the to a proximal end portion 306 of the body 304. Referring to FIG. 31, the retaining member 302 may be a cylindrical tube. In other alternatives the retaining member 302 may be C-shaped. In yet another alternative, the retaining member 302 could form the end of the package 300. In such an alternative, the retaining member 302 has a closed proximal end 330. The body of the package 300 would have an open proximal end. The retaining member 302 would attach to and close the open proximal end of the body of the package 300. In other words, the retaining member 302 would be a cap that closed the proximal end of the package. One or more projections 308 may project from the inner surface 310 and into the lumen 312 of the retaining member 302 (FIG. 30).

Referring to FIG. 30, during assembly the catheter product, the proximal end 23 of the proximal end section 22 of the catheter 20 may be inserted into the lumen 312 of the retaining member 302 (or the retaining member 302 may be placed over the proximal end 23) so that the one or more projections 308 enter and engages at least one eyelet 28 of the catheter 20. Optionally, the retaining member 302 may include a spacer 314, which may be a shoulder or protuberance, that extends from the inner surface 310 of the retaining member. When a spacer 314 is present, it urges the catheter 20 toward the projection 308 and assists in centering the catheter.

The catheter 20, having the retaining member 302 thereon, may be inserted through a distal opening in the package body 304 and advanced into the cavity 316 of the package body 304. The retaining member 302 is advanced into the body 304 until it is seated in a seat 318 near the proximal end 306 of the body 304. The seat 318 may form a friction/interference fit or a snap fit with the retaining member 302. In the illustrated embodiment, the seat 318 is defined by a proximal shoulder or lip 320 and a distal should or lip 322. Referring to FIG. 32, the retaining member 302 engages the seat 318 and sits between the proximal lip and distal lip 320 and 322. That is, the proximal end of the retaining member 302 engages the proximal lip 320 and the distal end of the retaining member 302 engages the distal lip 322. The lips 320 and 322 may extend partially or circumferentially around the body. Alternatively, the attaching of the retaining member 302 to the package 300 can be by any suitable attachment mechanism (glue, welding, snap/interference/friction fit, etc.) that holds the retaining member 302 in place while the catheter 20 is extended and removed from the package in a manner the same or similar to that described above.

When the retaining member 302 has a closed end and servers as the closed proximal end of the package 300, the retention member 302 can be attached to the body of the package 300. Then the catheter 20 can be inserted into the package.

FIGS. 33-35 illustrate another alternative package 400 and retaining member 402. In this embodiment, the body 404 of the package 400 includes an opening 406 into which the retaining member 402 is inserted. Referring to FIGS. 33 and 34, during assembly, the catheter 20 is inserted into the body 404 of the package 400 such that an eyelet 28 is aligned with the opening 406. The retaining member 402 is then inserted and positioned into the open so that one or more projections 408 of the retaining member 402 engages at least one eyelet 28 of the catheter 20, as shown in FIG. 34. For example, the projection 408 may have a free end the extends into the eyelet 28. Furthermore, the retaining member 402 may include a locking member 410 that engages the body 404 to lock the retaining member in the opening 406. In the illustrated embodiment, the lock 410 may be a flexible barb that bends to fit into the opening and then contacts the inner surface of the body. The package 400 may be gas and/or liquid impermeable and the retaining member may form a gas and/or liquid impermeable seal with the body. The package 400 may include a spacer 412, such as a protuberance or shoulder, to urge the catheter 20 toward the retaining member 402 and/or center the catheter within the package 400. The retaining member 402 retains the catheter 20 as it is extended and releases the catheter 20 as it is removed from the package 400 in a manner the same or similar to that described above. As shown in FIG. 35, after the catheter 20 has been extended, the projection 408 and/or the material of the catheter defining the eyelet 28 may flex or bend to disengage the projection from the eyelet thereby releasing the catheter from the retaining member and the package.

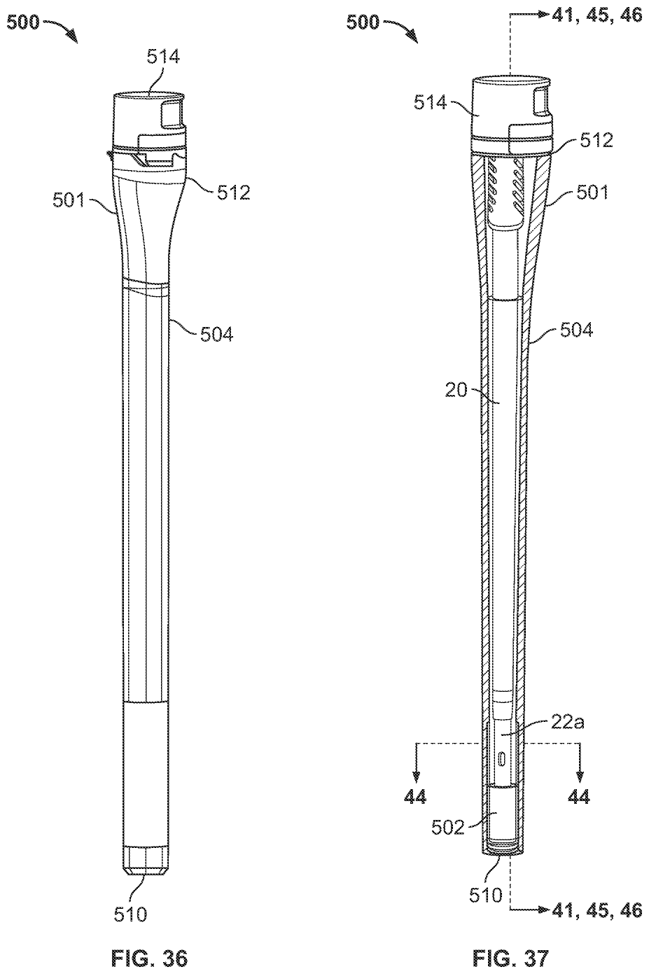

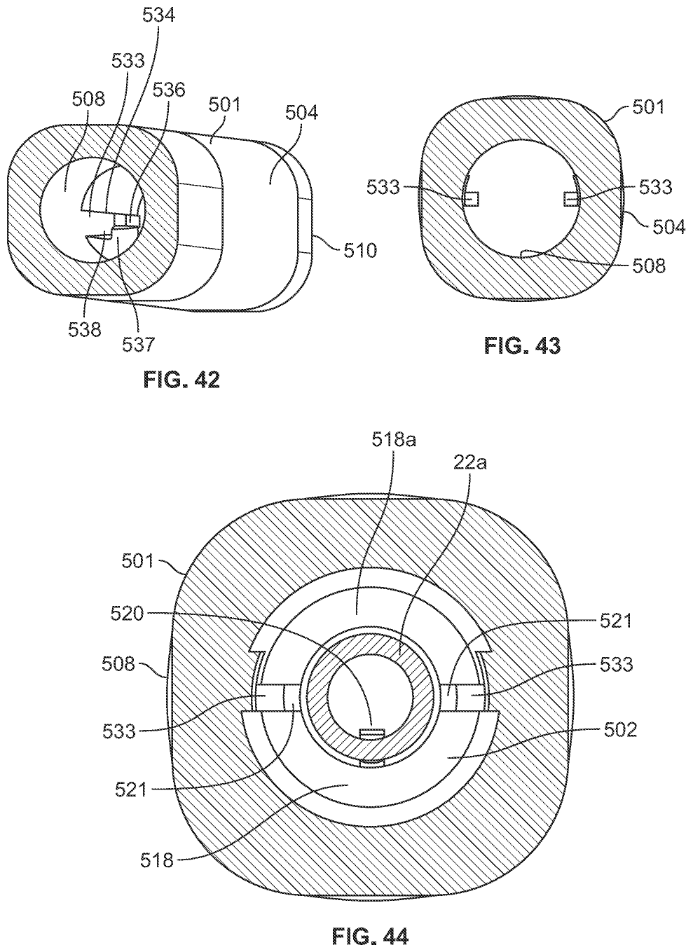

FIGS. 36-46 illustrate an alternative catheter product 500. Referring to FIGS. 36 and 37, catheter product 500 includes a package 501 that has a body 504. The body 504 has an internal cavity 506 (see in FIGS. 41, 45 and 46) defined by the inner surface 508 of the body. The body 504 also includes a closed proximal end 510 and an open distal end 512. An openable cap 514 closes the open distal end 512. Referring to FIGS. 37 and 40, catheter 20 and retaining member 502 are located within the cavity 506 of the package body 504. Catheter 20 is similar to the catheters disclosed above.

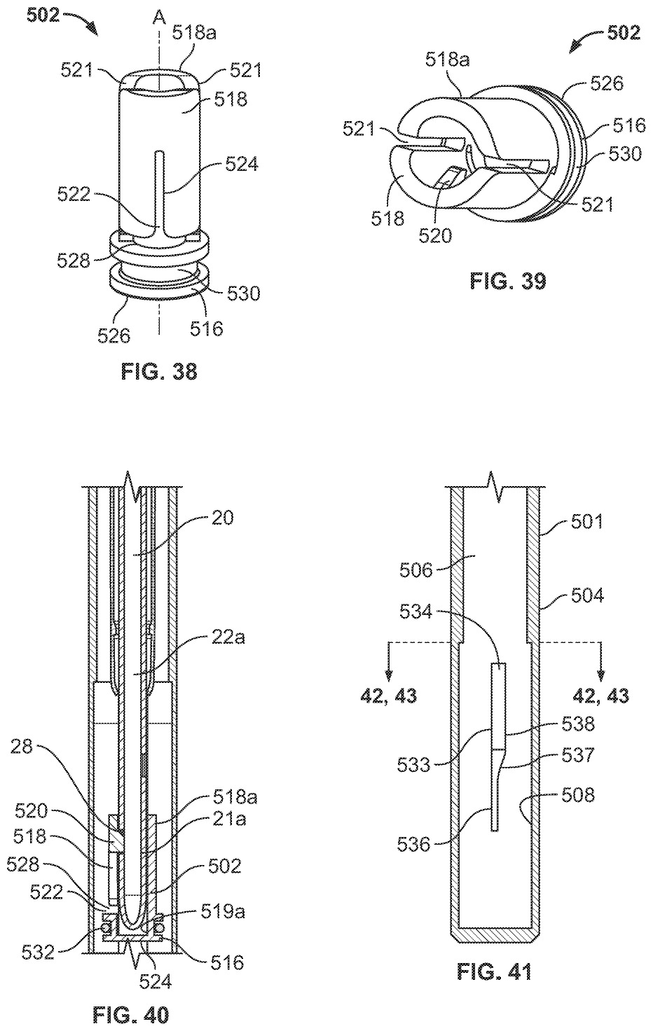

FIGS. 38 and 39 illustrate retaining member 502, which has a generally tubular shape. Retaining member 502 includes a proximal base 516 having two or more arms 518, 518a extending in a distal direction from the base. In the illustrated embodiment, the retaining member 502 has two arms 518, 518a. Alternatively, the retaining member 502 may have three, four or more arms. The arms 518, 518a are separated by slots 521. One or more of the arms 518, 518a includes a projection 520 for engaging an eyelet 28 of catheter 20. The arms 518, 518a have an arcuate cross-sectional shape and may be in a duck-billed configuration. One or more of the arms 518, 518a may include reliefs 522 that assist in allowing the arms 518, 518a to flex away from each other. In the illustrated embodiment, arm 518 has a first relief that includes a longitudinally extending slit 524 that extends in the direction of the longitudinal axis A of the retaining member or extends perpendicular to the plane of the terminal end 526 of the base 516. In addition to or in lieu of slit 524, arm 518 may include a relief in the form of a slit 528 that extends in a direction about the longitudinal axis of the retaining member 502. Optionally, the base 516 of the retaining member 502 includes a circumferential groove 530 that holds an O-ring 532 (FIGS. 40, 45 and 46). The O-ring 532 contacts the inner surface 508 of the package body 504. The O-ring 532 centers the retaining member 502. The force required to move the retaining member 502 within the cavity 506 may be varied by varying the size, flexibility and material of the O-ring 532.

Referring to FIG. 40, the proximal tube 22a of the catheter 20 is positioned between the arms 518, 518a of the retaining member 502. The projection 520 is engaged with the eyelet 28 of the catheter 20 to hold the proximal tube 22a. The space between the arms 518, 518a is a size that keeps the projection 520 engaged with the eyelet 28. That the distance between the projection 520 and the inner surface 519a of arm 518a is such that the portion 21a of the catheter 20 opposite the eyelet 28 contacts the inner surface 519a to maintain the engagement between the eyelet 28 and the projection 520.

Turning to FIGS. 41-44, the inner surface 508 of the package body 504 includes one or more spreaders 533 that spreads arms 518, 518a of the retaining member 502 as the retaining member 502 moves distally within the cavity 506 of the package body 504. The illustrated embodiment includes two spreaders 533 that are opposed and on opposite sides of the cavity 506 of the package body 504. The spreaders 533 are aligned and positioned within slots 521 between arms 518, 518a of the retaining member 502. The spreaders 533 are projections or tracks 534 on the inner surface 508 of the package body 504. The projections 534 include a proximal narrow segment 536 and a wide distal segment 538. Each projection 534 may be a single continuous projection/track or they may include segmented spaced apart sections with interruptions therebetween. Furthermore, the transition between the proximal narrow segment 536 and the wide distal segment 538 may be ramped or a gradual transition portion 537.

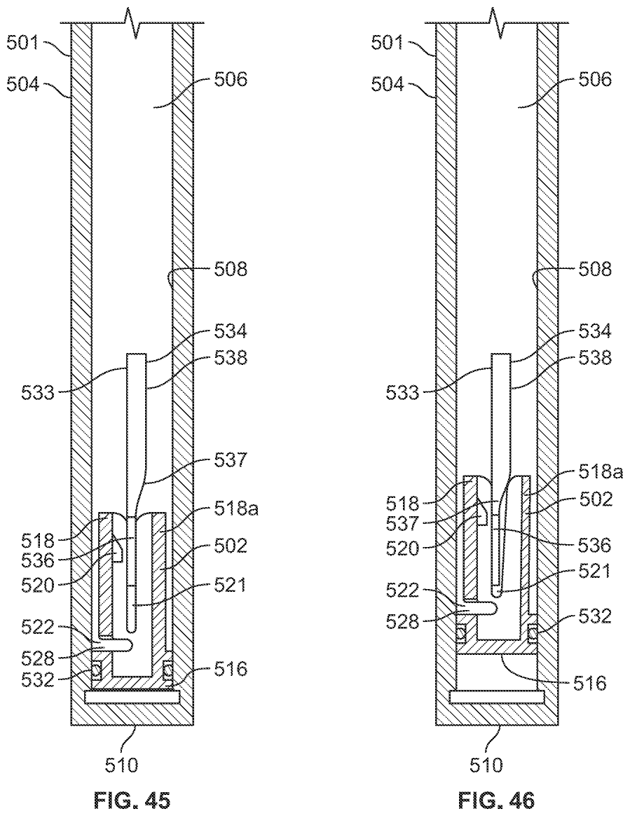

Turning to FIGS. 44 and 45, in the initial pre-opened configuration of the catheter product 500, the retaining member 502 is positioned within the cavity 506 of the package body 504 with the proximal narrow segments 536 of the spreaders 533 located within the slots 521 between arms 518, 518a of the retaining member 502. Additionally, the proximal tube 22a of the catheter 20 is located between the arms 518, 518a with the projection 520 engaged with eyelet 28. To remove the catheter 20 from the package 501, the user opens the cap 514 and grasps the distal end of the catheter 20. The user pulls the distal end of the catheter 20 to move the catheter 20 from the collapsed configuration to the expanded configuration. The engagement between the O-ring 532 and inner surface 508 of the package body 504 and/or the rigidity of the arms 518, 518a against the spreader 533 creates a counter-force that holds the retaining member 502 (holding the proximal tube 22a) in place within the package 501 as the distal tube and proximal tube move away from one another to place the catheter 20 in the expanded configuration and lock the locking mechanism in the locked configuration.

Referring to FIG. 46, once the catheter 20 is in the expanded configuration, the user continues to pull the distal tube from the package 501 with a greater force. This greater force overcomes the counter-force of the O-ring 532 and the arms 518, 518a against the spreaders 533, moving the retaining member 502 distally within the cavity 506 of the package 501. As the retaining member 502 moves distally, the side surfaces of the arms 518, 518a slide along the ramped transition 537 (if present) of the spreader 533 and move into contact with the wide segment 538 of the spreader 533. The contact between the wide segment 538 of the spreader 533 and the arms 518, 518a moves the arms away from each other, thereby allowing the eyelet 28 to disengage from the projection 520. The proximal tube 22a of the catheter 20 is released and the catheter is allowed to be removed from the package.

The holding force of the engagement between the retaining member and the distal end of the catheter and/or the eyelet is larger than the force needed to move the catheter into the expanded configuration and/or lock the catheter in the expanded configuration. Furthermore, the force needed to move the retaining member into a position along the spreader to allow the eyelet to disengage from the retaining member is larger than the force needed to move the catheter into the expanded configuration and/or lock the catheter in the expanded configuration. Additionally, when the retaining member is moved along the spreader, the holding force is reduced or released, allowing the catheter to be removed from the package.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A catheter product, comprising:
an extendable urinary catheter including a proximal end section and a distal end section that are concentric and slide relative to one another, the extendable urinary catheter having a collapsed configuration and an extended configuration;
a package containing the extendable catheter in the collapsed configuration, the package comprising a sidewall defining an elongated body having a cavity containing the catheter, wherein the sidewall extends over a majority of the proximal end section of the catheter and a majority of the distal end section of the catheter; and
a retaining member associated with a proximal end portion of the elongated body of the package, the retaining member holding the proximal end section of the extendable urinary catheter as the proximal and distal end sections of the extendable urinary catheter are pulled apart to slide the proximal and distal end sections relative to one another, thereby moving the extendable urinary catheter into the extended configuration.

2. The catheter product of claim 1, wherein the retaining member is configured to release the proximal end section of the catheter after the catheter has been moved into the extended configuration.

3. The catheter product of claim 1, wherein the retaining member includes a proximal base and two or more arms extending in a distal direction from the base, wherein the two or more arms include a projection engaged with an eyelet of the catheter.

4. The catheter product of claim 3, wherein the retaining member further includes slots between the two or more arms, and the package includes an inner surface having spreaders located in the slots.

5. The catheter product of claim 4, wherein the spreaders comprise tracks on the inner surface of the package and the tracks are located within the slots.

6. The catheter product of claim 5, wherein the tracks include a narrow segment and a wide segment, wherein contact between the wide segment and the arms spreads the arms apart to release the projection of the arm from the eyelet of the catheter.

7. The catheter product of claim 6, wherein at least one of the two or more arms includes a relief.

8. The catheter product of claim 4, wherein the retaining member includes an O-ring in contact within the inner surface of the package.

9. The catheter product of claim 1, wherein the retaining member comprises a flexible projection releasably engaged with an eyelet of the catheter.

10. The catheter product of claim 9, wherein the flexible projection is generally cone-shaped or hook-shaped.

11. The catheter product of claim 9, wherein the retaining member comprises a collar defining a lumen containing a proximal end of the proximal end section of the catheter and the projection extends into the lumen of the retaining member.

12. The catheter product of claim 11, wherein the collar is c-shaped or ring-shaped.

13. The catheter product of claim 1, wherein the retaining member is fixed to the proximal end portion of the elongated body of the package.

14. The catheter product of claim 13, wherein the retaining member is engaged with a seat of the elongated body of the package.

15. The catheter product of claim 13, wherein the retaining member is positioned in an opening of the elongated body of the package.

16. The catheter product of claim 13, wherein the retaining member extends from an inner surface of the elongated body.

17. The catheter product of claim 1, wherein the elongated body of the package has a top portion and a bottom portion that are attached to one another to form the cavity of the elongated body.

18. The catheter product of claim 9 wherein the projection has sufficient rigidity to maintain engagement with the eyelet while the catheter is being moved into the extended configuration and sufficient flexibility to bend and disengage the eyelet after the catheter is extended and is being pulled from the package.

19. A catheter product, comprising:
an extendable urinary catheter including a proximal end section and a distal end section that are concentric and slide relative to one another, the extendable urinary catheter having a collapsed configuration and an extended configuration;
a package defining a body that contains the extendable catheter in the collapsed configuration; and
a retaining member releasably engaged to the proximal end section of the catheter, the retaining member being movable axially within the body; and
a stop located in the body, the retaining member engaging the stop as the catheter is pulled from the body of the package to move the catheter into the extended configuration.

20.-25. (canceled)

26. A catheter product, comprising:
an extendable urinary catheter including a proximal end section and a distal end section that are concentric and slide relative to one another, the extendable urinary catheter having a collapsed configuration and an extended configuration;
a package having a body defining a cavity that contains the extendable catheter in the collapsed configuration; and
a flexible retaining member within the body, the flexible retaining member comprising one or more projections having a free end engaging at least one eyelet in the proximal end section of the extendable urinary catheter and holding the proximal end section of the extendable urinary catheter as the proximal and distal end sections of the extendable urinary catheter are slid relative to one another to move the extendable urinary catheter into the extended configuration.

27.-42. (canceled)
